(19) United States
(12) Patent Application Publication
(10) Pub. No.: US 2007/0225653 A1
(43) Pub. Date: Sep. 27, 2007

(54) Material dispenser with a solenoid lock

(75) Inventors: Bernard C.B. Lim, Oakville (CA); David G. Matsuura, Encinitas, CA (US); Philip J. Simpson, Escondido, CA (US); Mark Costa, Milton (CA); Hao Chen, Mississauga (CA); Kathleen Chancellor-Maddison, Hamilton (CA); Davis A.R. Kanbergs, Milton (CA)

Correspondence Address: BUCHANAN, INGERSOLL & ROONEY PC, POST OFFICE BOX 1404, ALEXANDRIA, VA, 22313-1404, US

(73) Assignee: Vasogen Ireland Limited, Shannon (IE)

(21) Appl. No.: 11/640,202

(22) Filed: Dec. 18, 2006

Related U.S. Application Data
(60) Provisional application No. 60/752,378, filed on Dec. 22, 2005.

(51) Int. Cl. A61M 5/00

(52) U.S. Cl. 604/187

(57) ABSTRACT

A syringe including a releasable lock means for allowing discharge of a treated biological fluid sample to the patient in response to a release signal to the releasable lock means. The release signal is issued following a positive outcome from a verification process dependent upon temporal data from certain events in the collection, treatment and delivery of the biological fluid sample, and identity data of the patient and the second device with the treated biological fluid.

### CROSS-REFERENCE TO RELATED APPLICATIONS

This application claims the benefit of priority to U.S. Provisional Application Ser. No. 60/752,378, filed Dec. 22, 2005.

### BACKGROUND OF THE INVENTION

1. Field of the Invention

The present invention relates to the management of medical treatments, more specifically it relates to a permission-based fluid dispensing device.

2. Description of the Prior Art

Despite remarkable advances in health care technology and delivery, a large number of patients die or are disabled as a result of medical errors. These errors occur in health care settings, such as hospitals, clinics, nursing homes, urgent care centers, physicians' offices, pharmacies, and the care delivered in the home, and they usually result from systems problems rather than one single action or decision.

For many years, bar code labelling has been the technology of choice in ensuring patient safety. Recently, the Food and Drug Administration (FDA) issued a new rule which requires certain human drug and biological product labels to have bar codes. As such, the bar code for human drug products and biological products (other than blood, blood components, and devices regulated by the Center for Biologics Evaluation and Research) must contain the National Drug Code (NDC) number in a linear barcode. The rule is geared toward reducing the number of medication errors in hospitals and other health care settings by allowing health care professionals to use bar code scanning equipment to verify that the right drug (in the right dose and right route of administration) is being given to the right patient at the right time. The rule also requires the use of machine-readable information on blood component container labels to help reduce medication errors.

However, bar codes require line of sight with a reader in order to be read and they cannot store additional information apart from simple identification data, such as a serial no. or a SKU. For example, a bar-coded wristband on a patient is not easy to read if the patient gets it wet or is sleeping on top of the arm bearing the wristband, or when the patient is on an emergency room gurney or operating table; these are instances where mistakes in medication or blood transfusion are most prevalent.

It is an object of the present invention to mitigate or obviate at least one of the above-mentioned disadvantages.

### SUMMARY OF THE INVENTION

In one of its aspects, the present invention provides a syringe for treating a patient in a biological fluid treatment system, the patient having a patient identifier, the syringe comprising:
a syringe inlet operable to form a fluid coupling with a biological fluid treatment chamber outlet;
a syringe chamber for receiving the treated biological fluid;
a syringe outlet;
a passage in communication with the chamber and the syringe outlet;
an incremental counter for recording temporal data corresponding to biological fluid treatment events, treated biological fluid events and delivery events; the syringe being associated with a unique identifier, the unique identifier correlatable to the patient identifier;
a releasable lock means having a biased blocking member within the passage and a solenoid assembly with a biased solenoid core engaging the blocking member to maintain the outlet in a closed position;
a verification means having:
   a comparator for comparing the unique identifier to the patient identifier to confirm the correlation between same;
   a computer readable medium for memory means for storing the unique identifier, the patient identifier, temporal data, and data related to biological fluid treatment events, treated biological fluid events and delivery events;
   a logic means for receiving the temporal data to determine at least one time period between the events and for determining whether the at least one time period is within a predefined range;
   a release signal generating means for issuing a release signal to the releasable lock means upon positive confirmation of the correlation between the patient identifier and the unique identifier, and provided that the at least one time period is within a predefined range;
whereby the solenoid assembly is actuated in response to the release signal to disengage the biased solenoid core from the biased blocking member, thereby placing the syringe outlet in an open position.

In another of its aspects, the present invention provides a method for processing of a first patient material sample for administering to a second patient, comprising the steps of: equipping the first patient with a first machine-readable patient identifier; equipping the second patient with a second machine-readable patient identifier; providing a first device for receiving the first patient material sample, the first device having a machine-readable first device identifier; transferring the first patient material sample to at least one treatment device; enabling at least one treatment device to carry out a treatment of the first patient sample material; providing a second device for receiving the treated first patient material sample, the second device having a machine-readable machine-readable second device identifier; providing at least one correlation unit, recording temporal data associated with said steps of said processing, and corresponding to first patient material sample treatment events; enabling the correlation unit to perform a correlation function between the first patient machine-readable identifier, the machine-readable second patient identifier, the machine-readable first device identifier; and the machine-readable second device identifier; enabling the correlation unit to analyze said temporal data to determine whether said steps occur within predetermined time periods; whereby the second device delivers the treated first patient material sample following a correlation between the first patient machine-readable identifier, the machine-readable second patient identifier, the machine-readable first device identifier; and the machine-readable second device identifier; and provided that said steps occur within predetermined time periods.

### BRIEF DESCRIPTION OF THE DRAWINGS

These and other features of the preferred embodiments of the invention will become more apparent in the following detailed description in which reference is made to the appended drawings of the specific, most preferred embodiment of the invention, wherein:

FIG. 1 is a perspective view of a syringe with an inlet end cap and an outlet end cap;

FIG. 2 is another perspective view of the syringe and associated RFID reader;

FIG. 3 is a sectional view of the syringe of FIG. 2 taken along line 3-3′;

FIG. 4 is a perspective view of a solenoid assembly used in the syringe of FIG. 3;

FIG. 5 is a partial exploded view of the syringe of FIG. 1;

FIG. 6 is a perspective view of the syringe with a needle coupled thereto, and associated wristband;

FIG. 7 is sectional view of a valve outlet means of FIG. 5 taken along line 7-7′;

FIG. 8 is a view of a releasable locking means incorporating the solenoid assembly of FIG. 4;

FIG. 9 is a view of the proximal end of the syringe of FIG. 1;

FIG. 10 is a flowchart outlining the steps for a verification protocol; and

FIG. 11 is a schematic diagram of a verification means employed in the verification protocol.

### DESCRIPTION OF THE PREFERRED EMBODIMENTS

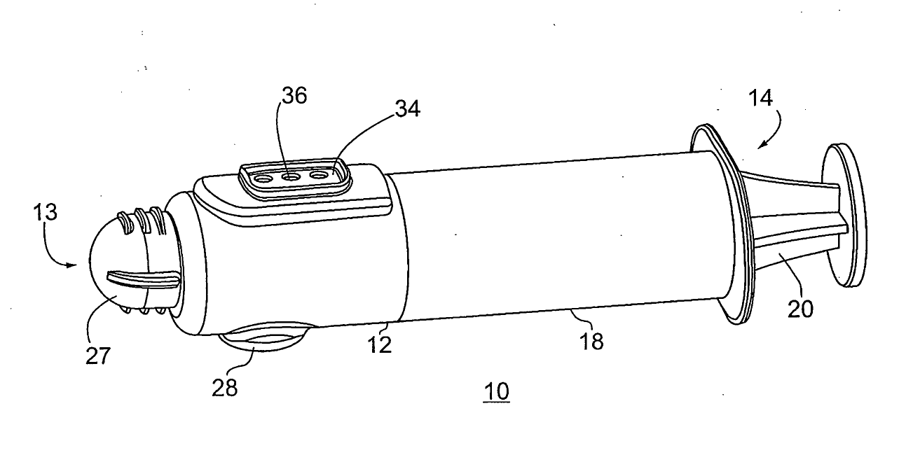

As shown FIG. 1, there is provided a syringe 10 to receive a biological fluid sample, such as a blood sample, in biological fluid treatment system to treat Generally, the biological fluid treatment system includes a plurality of entities which are used at different stages during the handling of the blood sample, such as, a blood collection syringe to withdraw an untreated blood sample from a patient, a blood sample blood treatment chamber, a blood treatment unit, a blood delivery syringe 10, and a patient identifier, such as wristband with a tag. Following collection of the untreated blood sample, the blood collection syringe is coupled to the blood treatment chamber, and the sample is delivered to the blood treatment unit, in which the untreated blood sample is subjected to one or more stressors, such as ozone or an ozone/oxygen mixture, ultra-violet (UV) light and infra-red (IR) energy.

Following treatment, the treated blood sample is delivered to the blood delivery syringe 10, from which the treated blood sample is administered to the patient. At one or more stages in the treatment process, the system provides for a verification check, aimed at reducing the risk of error, and thus ensuring that the correct blood sample is returned to the correct originating patient. The verification check includes the steps of matching the blood sample, either in its treated or untreated form or both, with the originating patient.

Below is a description of the post-treatment portion of the blood treatment process involving the use of the syringe 10 which ensures that the correct blood sample is returned to the correct originating patient. As shown in FIGS. 1, 2 and 3, the syringe 10 includes a body portion 12 with a proximal end 13 and a distal end 14. Disposed at the proximal end 13 are an inlet port 15 and an outlet port 16. The syringe body portion 12 has a cylindrical cavity 18 which in cooperation with a plunger 20 provides a sample receiving chamber 21. The inlet port 15 is disposed at an angle to the outlet port 16, and intermediate the sample receiving chamber 21 and the outlet port 16. The plunger 20 is slidably disposed at the distal end 14 and is in tight fluid engagement with the wall defining the cylindrical cavity 18. The plunger 20 serves to draw fluid into the chamber 21 and urge the fluid therefrom. The syringe 10 also includes a channel portion 22 with a channel 24 in communication with the chamber 21 and the outlet port 16, and a channel 26 in communication with the inlet port 15 and the chamber 21 via a portion of the channel 24. The channel portion 22 is separated from the proximal end 13, or nose portion by a collar 25.

In order to prevent large particulate from entering the outlet port 16, an end cap 27 may be removably attached thereto, while the inlet port 15 may include a cap 28 to prevent contamination prior to use with the blood treatment unit. The treated blood sample is dispensed from the syringe 10 to the originating patient via the syringe outlet port 16 operable between an open position and a closed position by a releasable locking means, such as a solenoid locking means 30, as will be described below.

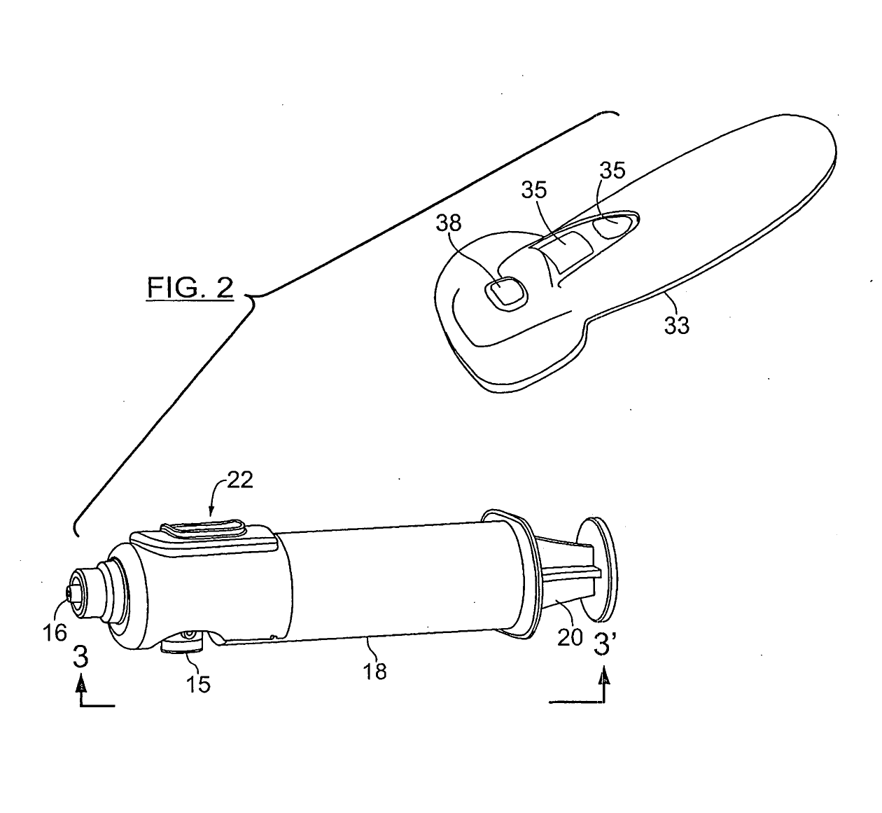

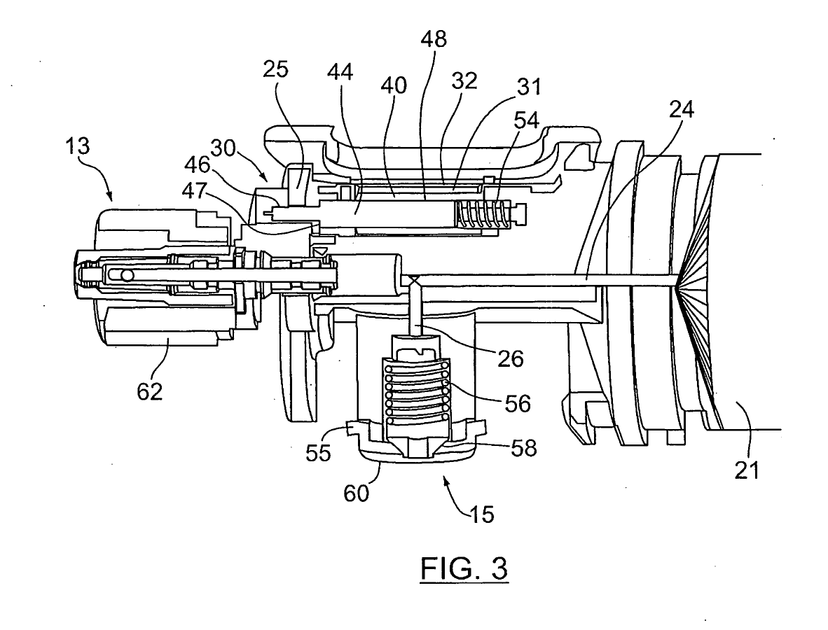

As shown in FIG. 3, within the channel portion 22 is a printed circuit board (PCB) 31. On one side of the PCB 31 is circuitry for transmitting, receiving and storing data related to the syringe 10 and/or its contents or the originating patient. The circuitry includes a radio identification (RFID) integrated circuit with reader/writer functionality coupled to an antenna, such as an RFID tag 32. As such, the circuitry includes, but is not limited to, a transmitter, a receiver, logic means or processor, a computer readable medium, a timing circuit, an antenna and a power source. Also coupled to the PCB 31 are input/output devices such as a display, LED, a speaker or a button. The RFID tag 32 is interrogated by an RFID reader 33. A recessed compartment 34 provides an interface for the syringe 10 such that the syringe 10 is coupled to the reader 33, as shown in FIG. 2. The reader 33 includes a user interface 35, such as LCD display or LEDs to provide visual signals to a user. The visual signals may include instructions or alerts for the user. Alternatively, auditory signals may be issued via a speaker included in the reader 33. As such, the PCB 31 also includes electrical contacts 36 for interfacing with corresponding reader contacts 38. On the opposing side of the PCB 31 is a solenoid 40 which is part of the locking mechanism 30. The electrical contacts 36 are coupled to a solenoid coil 50 to provide energizing power thereto from the reader 33.

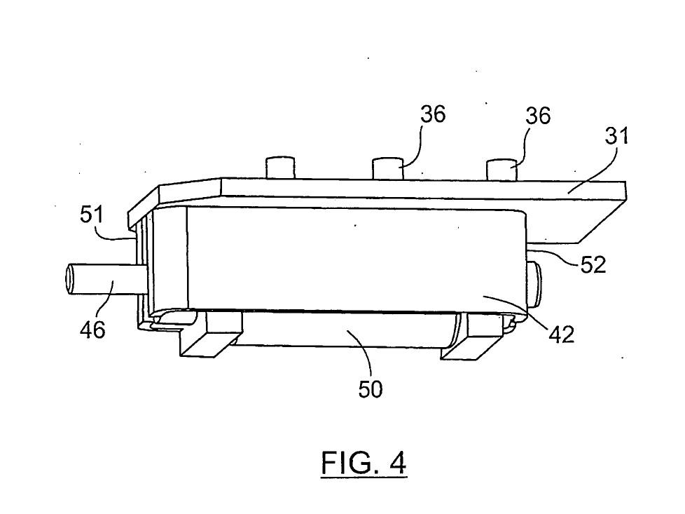

As shown in FIGS. 3 and 4, the solenoid assembly 40 includes a solenoid frame 42 with a cylindrical solenoid core 44, with a reduced-diameter solenoid core portion 46 formed from a circumferential face 47 of the solenoid core 44. The reduced-diameter solenoid core portion 46 received by a solenoid spool passageway 48 having the solenoid coil windings 50. The solenoid core 44 is free to travel along the spool passageway such that the reduced-diameter solenoid core portion 46 extends from one end 51 of the solenoid frame 42 in a rest position, and retracts towards the other end 52 when the solenoid coil 50 is energized. A resilient means, such as spring 54 biases the circumferential face 47 against the inside of the solenoid frame at end 51 in the rest position. As such, solenoid core 44 is contained between the two ends 51 and 52.

As shown in FIG. 3, the syringe inlet port 15 includes bayonet pins 55 extending outwardly therefrom, which engage complementary grooves in a collar portion of a blood treatment chamber receptacle for coupling thereto. Similarly, a valve element 56 is located in the channel 26 and biased to a closed position against a valve seat 58 on a valve cap 60 forming the outer end of the syringe inlet port 16. The valve element 56 is also aligned for abutment with a valve actuating element which is positioned in the chamber receptacle (not shown). The valve actuating element is thus operable to displace the valve element 56 from its closed position against the valve seat 58 to allow fluid flow therethrough.

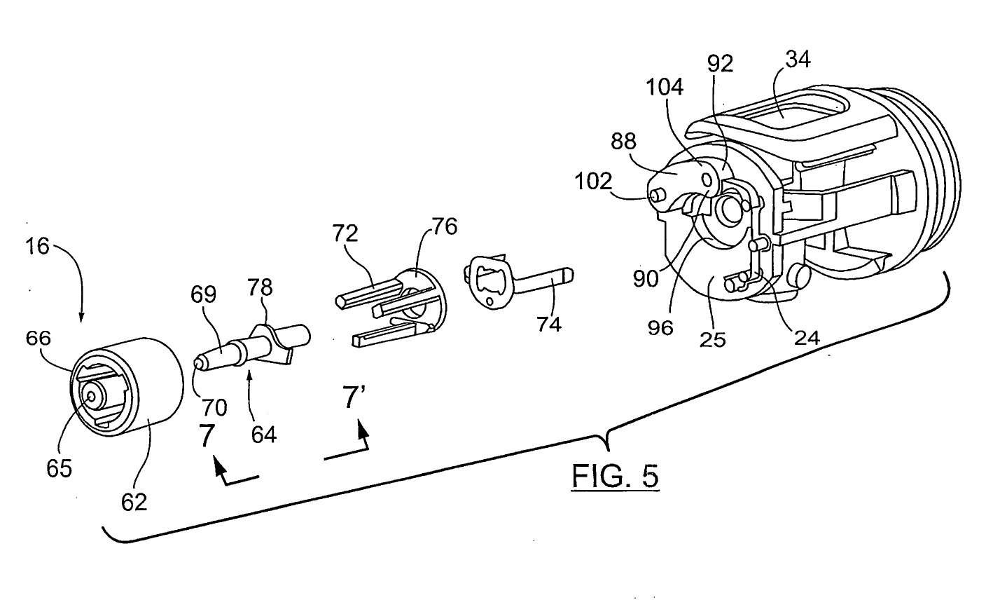

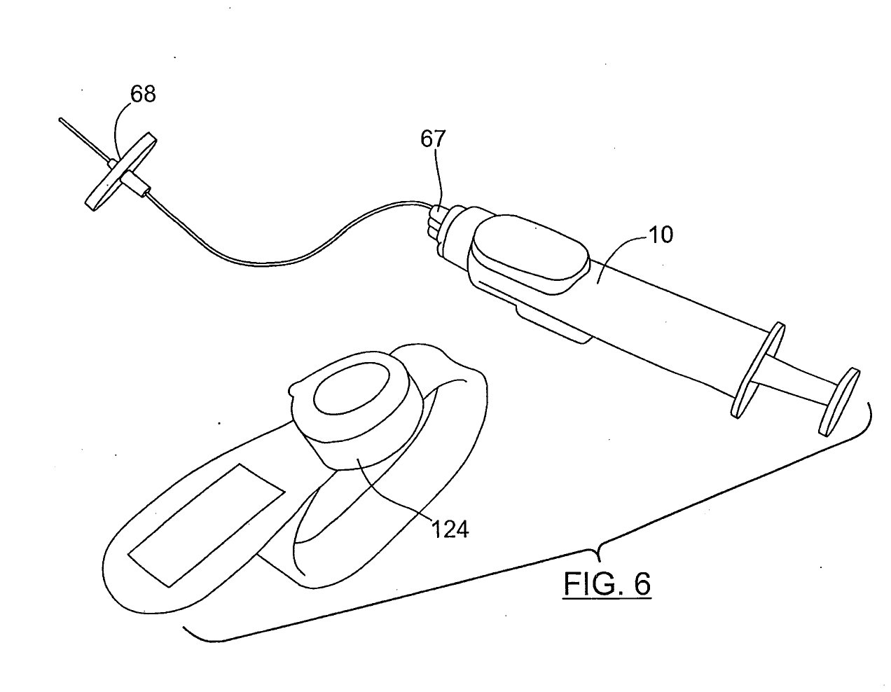

As best shown in FIG. 5, the syringe outlet port 16 includes a male Luer insert 62, an outlet valve means generally shown at 64 for opening and closing the access to the fluid channel 24 to control the flow of the blood sample therethrough. The male Luer insert 62 includes an opening 65, a valve seat 66 adjacent thereto, and a thread for the Luer fitting for coupling with a corresponding female Luer 67 of a medical accessory, such as needle 68, as shown in FIG. 6. The outlet valve means 64 includes a valve element portion 69, a valve seat portion 70, and actuating elements generally shown at 72 for actuating the valve element portion 69 relative to the valve seat 66. A pair of resilient members 74, such as a spring, biases the outlet valve means 66 in a closed position. As will be described, the actuating means 72 is operable to displace the valve element portion 69 in opposed directions when the Luer 62 portion 20 is engaged or disengaged with a female Luer 67.

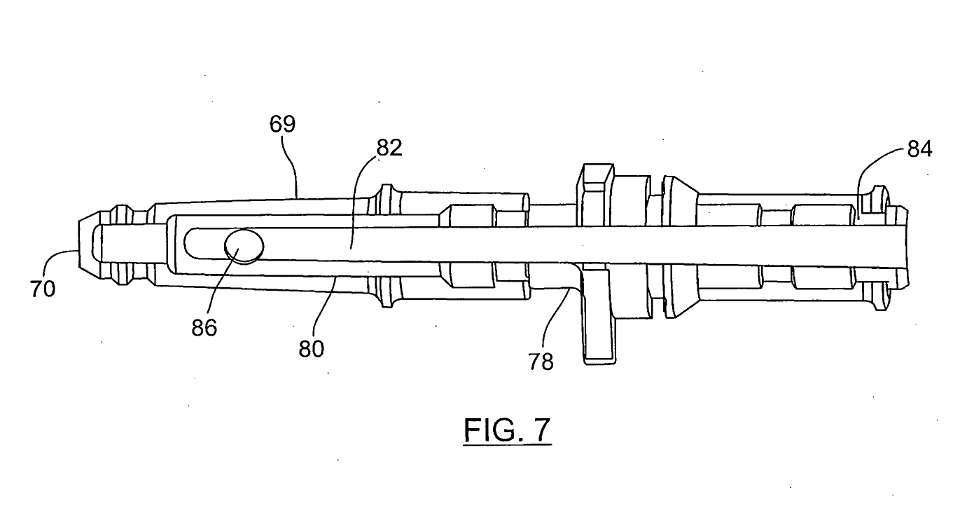

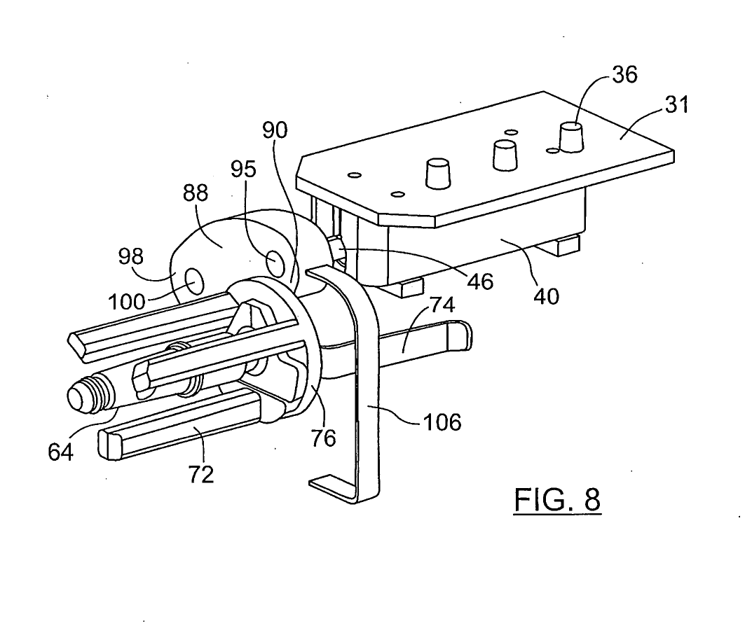

As shown in FIG. 7 and 8, the actuating elements 72 extend outwardly from a central web 76 fixed to a block 78 positioned in the channel 24 in the body portion 22 of the syringe 10. The block 78 has a central bore 80 carrying a tubular valve stem 82 having one end carrying the valve element portion 69 and an opposite end carrying a valve stem head 84, which has a peripheral edge region with a sealing element such as an O-ring or the like. The valve stem 82 has a pair of fluid transfer holes, as shown at 86, immediately beside the valve element portion 69, thereby forming an inner valve passage in fluid communication with the chamber 21. The female Luer 67 includes complementary actuating elements which displace the actuating elements 72, when the female Luer 67 member is introduced into the male Luer insert 62. Subsequently, the actuating elements 72 displace the valve stem 82 and the valve element portion 69 to open the central bore 80 within the valve stem 82 to the channel 26 to allow fluid flow through outlet port 16. The treated blood sample is dispensed from the syringe 10 to the originating patient via the syringe outlet port 16 operable between an open position and a closed position by the locking mechanism 30, as will now be described.

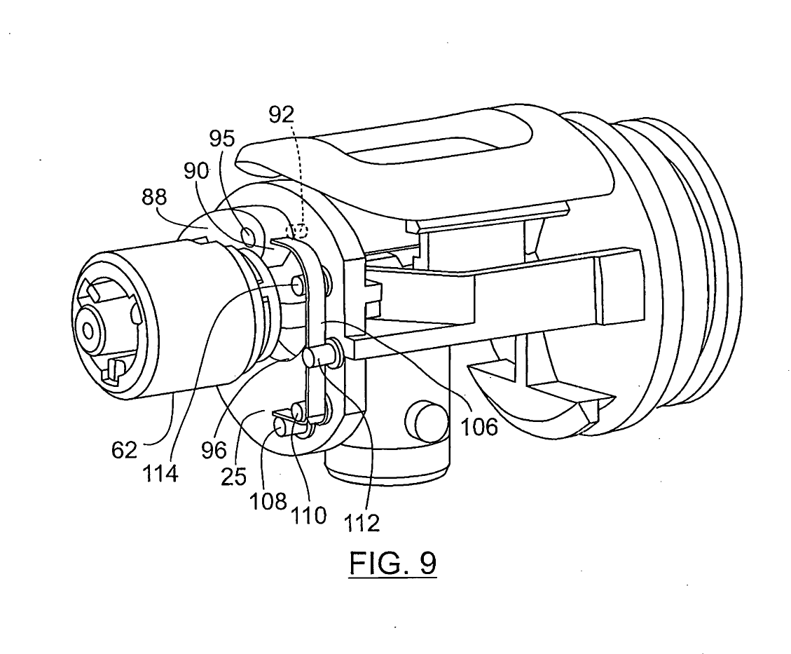

The outlet port 16 is operable between three states, a locked state, an open state and a permanent disabled state by the locking mechanism 30. The locking mechanism 30 controls the coupling of the female Luer 67 to the male Luer insert 62 of the syringe 10. As shown in FIGS. 5, 8 and 9, the locking mechanism 30 comprises the solenoid 40 and a locking pawl 88, which co-operate to lock the syringe 10. The solenoid assembly 40 and the locking pawl 88 are positioned on either side of the collar 25. The locking pawl 88 has a pawl portion 90 disposed within the passage 24 to prevent longitudinal displacement of the central web 76 away from the proximal end 13. The collar 25 includes an opening 92 which receives the reduced-diameter solenoid core portion 46. The thickness of the collar 25 is so dimensioned such that the reduced-diameter solenoid core portion 46 extends to the other side of the collar 25 to engage the locking pawl 88 via an opening 95 therethrough, in the rest position or locked state. The collar 25 also includes a passage 96 dimensioned to allow free travel of the central web 76 to and from the proximal end, as will be described later. As shown in FIG. 8, the locking pawl 88 includes one end 98 with an opening 100 for receiving a pivoting pin 102 protruding perpendicularly from the collar 25. The other end 104 of the locking pawl 88 includes the other opening 95, and a leaf spring 106 which is biased to cause anticlockwise rotation of the locking pawl 88 about the pivoting pin 102. However, since the reduced-diameter solenoid core portion 46 engages the opening 95 such motion is precluded, until the reduced-diameter solenoid core portion 46 retracts. The leaf spring 106 is held in place by a plurality of pins 108, 110, 112 and 114 protruding from the collar 25, the pins 108, 110, 112 and 114 have narrow recesses to receive and anchor the leaf spring 106.

Any attempt to couple a female Luer 67 fails, since the complementary first actuating elements cannot displace the actuating elements 72, and therefore the female Luer 67 and male Luer insert 62 cannot mate. Correspondingly, the outlet valve means 64 is biased closed by the pair of resilient members 74 acting on the central web 76, and thus the central bore 80 within the valve stem 82 is closed.

Upon receipt of the release signal following the verification process, an energizing current is sent to the solenoid coil 50 which acts to attract the solenoid core 44 thus causing the solenoid core 44 to retract. Typically, the energizing current is received from an external means, such as the reader 33. However, the energising source can be onboard the syringe 10, such as batteries. With the solenoid core 50 retracted, the leaf spring 106 forces the locking pawl 88 to swing anticlockwise about the pivoting pin 102, and the passage 24 is now free of the pawl portion 90. This places the syringe 10 in an open position. With the locking mechanism 30 unlocked, the syringe outlet port 16 is operable to form fluid coupling with a fluid fitting on a common blood sample delivery unit with the complementary female Luer 67 or similar fitting, such as the needle 68.

The central web 76 can now travel through the collar passage 96, in sympathy with the force on the actuating elements 72 the treated blood is expressed from the chamber via the open outlet valve into the patient, as shown in FIG. 7. The female Luer 67 can now be introduced into the male Luer insert 62. As such, the complementary first actuating elements abut the actuating elements 74 and the force applied to mate the female Luer 67 to the male Luer insert 62 displaces the actuating elements 72 and the central web 76 towards the opening 96. The force applied to couple the Luers 62 and 67 is sufficient to compress the resilient members 74 and thus open the central bore 80 within the valve stem 82.The energizing current flow to the solenoid 40 is turned off from the coil 50 once the locking pawl 88 has been released. The Luers 62, 67 are thus successfully coupled.

As the treated blood often includes bubbles of gases used during treatment, therefore, the syringe 10 includes a de-bubbling system or bubble removal mechanism to expel gas from syringe. Alternatively, a separate vent cap is attached to the proximal end 13 to interface with the LUER 50. The vent cap includes a hydrophobic gas permeable membrane to prevent blood from escaping. Generally, more air can be introduced into the chamber 21 to coalesce the existing bubbles, thus facilitating removal of otherwise small bubbles. Thus, the wall that defines the cylindrical cavity 18 is transparent such that a user can inspect the treated blood sample to verify that gas bubbles have been removed, after which the treated blood sample is ready for administration to the originating patient.

After the treated blood has been administered to the patient, the female Luer 67 is uncoupled from the male Luer insert 62, as the needle 68 is removed. With the energizing current is removed from the solenoid coil 50, the reduced-diameter solenoid core portion 46 is forced out of the solenoid frame 42 by the spring 54. However, the pawl opening 95 is now misaligned with the collar opening 92 and so the reduced-diameter solenoid core portion 46 no longer engages the pawl opening 95. Consequently, the locking pawl 88 can not forced to return to the rest position due to the action of the leaf spring 106, and thus the syringe 10 can not be locked again.

With the complementary actuating elements removed from the male Luer insert 62, the resilient members 74 expand to push the central web 76 towards the opening 65 and the central web 76 is forced out of the passage 96. The RFID tag 32 is disabled following the verification process, and subsequent use of the syringe 10 is precluded, thus substantially eliminating contamination risks.

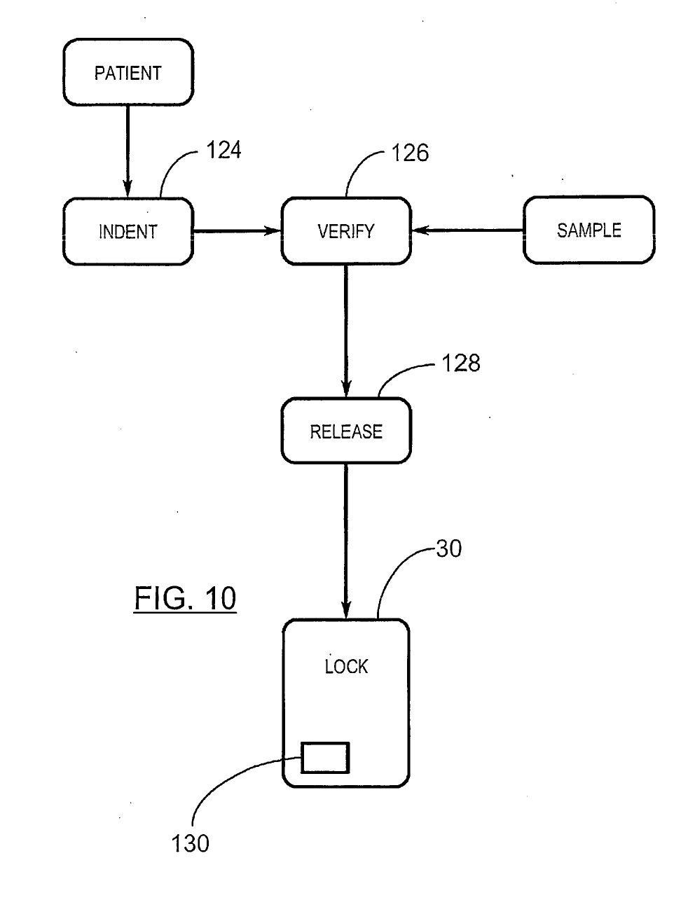

As previously mentioned, the system includes a verification protocol which includes a number of verification checks to ensure that the correct treated blood sample is delivered to the correct originating patient, and that certain events in the collection, treatment and delivery of the blood sample to the patient occur within prescribed time periods. To that end, and as shown in FIGS. 5, 10 and 11, the verification protocol includes identification means (Ident) 124, such as a wristband with identification means, such as an RFID tag, for identifying an originating patient, and the treated blood sample in the syringe 10, verification means 126 for verifying a match between the originating patient, and the treated blood sample in syringe 10, and release signal generating means 128 for generating a release signal in response to a positive verification by the verification means. The release signal is conveyed to the releasable locking mechanism 30 to deliver the predetermined current to the solenoid 40, thereby to render the syringe 10 operable to deliver the treated blood sample to the originating patient. The releasable locking mechanism 30 has a signal receiving means 130 for receiving the release signal. A visual signal indicative of a successful match or unsuccessful match may also be displayed on the interface 35, an auditory signal may be issued.

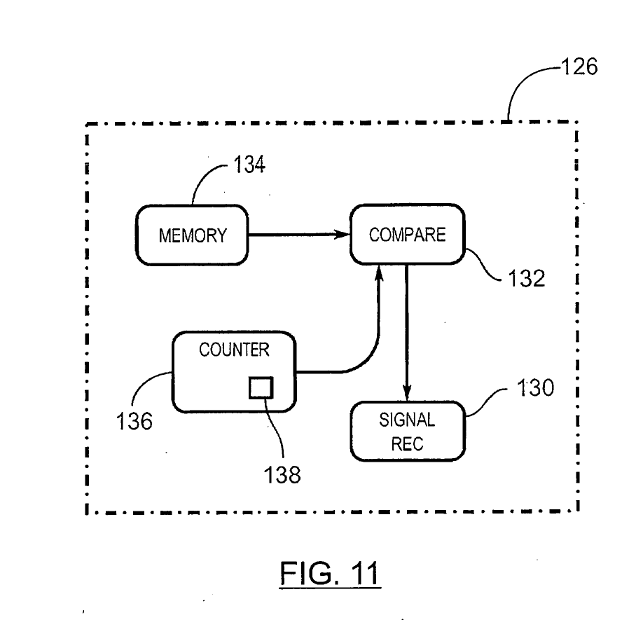

As shown in FIG. 11, the verification means 126 includes comparison means 132 for comparing patient identity data with treated blood sample identity data, both stored in memory means 134, and signal receiving means 130 to receive one or more signals associated with the originating patient identity data and/or the blood sample identity data. In this case, the one or more signals contain the originating patient identity data and/or the blood sample identity data. However, as an alternative, the one or more signals may contain data which is associated with or related to the patient or blood sample identity data. For example, the data in the signals may include one or more codes which allow the patient identity data or the blood sample identify data to be obtained from a data structure in the memory means 134 or some other location, for example in the form of a look-up table.

The verification means 126 may also include counter means 136 which provides temporal data related to a predetermined event including and/or between an untreated blood sample collection event and a treated blood sample delivery event. The temporal data may also include at least one elapsed time value between predetermined events related to an untreated blood sample collection event, a blood sample treatment event, or a treated blood sample delivery event. The counter means 136 may be implemented as an incremental counter 138 or a real-time clock. In this case, the incremental counter 138 tracks the events related to the treatment and post treatment events.

Before treatment of the untreated blood sample, the verification means 126 is also operable to prevent treatment of the blood sample if the elapsed time value following the blood withdrawal from the patient has exceeded a predetermined value. Post-treatment, the verification means 126 issues an appropriate signal to the releasable locking means 30 to prevent opening of the syringe outlet 16 when the elapsed time value has exceeded a predetermined value. Also, the verification means 126 is operable to verify an identity match between the untreated blood sample in the syringe 10 and the originating patient, or a correlation between the identity data of same.

The syringe may be used in a system for the collection, treatment and delivery of an autologous blood sample. For example, the syringe may be used to receive a treated blood sample delivered to a syringe 10 which then is used to deliver the treated sample for injection into the originating patient. The syringe 10 then verifies whether the treated blood sample was withdrawn from originating patient, and a release signal is provided to the locking mechanism 30 to allow discharge of the treated blood.

In another embodiment, the identification means, verification means and/or the release signal generating means may be located on other entities of the system 10. For example, verification means and/or the release signal generating means may be located on the wristband, or on the blood sample transfer portion 22, the reader 33, or the blood treatment unit.

The invention may be used with other autologous samples other than blood samples, such as bone marrow, lymphatic fluids, semen, ova-fluid mixtures, human milk, other bodily fluids or other medical fluids, for example fluid mixtures perhaps containing a patient desired solid sample such as from organs, body cells and cell tissue, skin cells and skin samples, spinal cords. Alternatively, the invention may be used with non-autologous samples such as in donor-recipient situations, in which it is a requirement that the intended recipient receives the correct sample from the correct donor, such as, blood banks or human milk banks, bone marrow transplant, stem cell transplants, umbilical cord transplants or other organ transplants. The syringe 10 may also be used for medical testing where it is important to ensure that test results of a particular test can be delivered to the originating patient.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

The embodiments of the invention in which an exclusive property or privilege is claimed are defined as follows:

1. A syringe to provide treatment to a patient in a biological fluid treatment system, the patient having a patient identifier, the syringe comprising:
a syringe inlet operable to form a fluid coupling with a biological fluid treatment chamber outlet;
a syringe chamber for receiving the treated biological fluid;
a syringe outlet;
a passage in communication with the chamber and the syringe outlet;
an incremental counter for recording temporal data corresponding to biological fluid treatment events, treated biological fluid events and delivery events; the syringe being associated with a unique identifier, the unique identifier correlatable to the patient identifier;
a releasable lock means having a biased blocking member within the passage and a solenoid assembly with a biased solenoid core engaging the blocking member to maintain the syringe outlet in a closed position;
a verification means having:
a comparator for comparing the unique identifier to the patient identifier to confirm the correlation between same;
a computer readable medium for memory means for storing the unique identifier, the patient identifier, temporal data, and data related to biological fluid treatment events, treated biological fluid events and delivery events;
a logic means for receiving the temporal data to determine at least one time period between the events and for determining whether the at least one time period is within a predefined range;
a release signal generating means for issuing a release signal to the releasable lock means upon positive confirmation of the correlation between the patient identifier and the unique identifier, and provided that the at least one time period is within a predefined range;
whereby the solenoid assembly is actuated in response to the release signal to disengage the biased solenoid core from the biased blocking member, thereby placing the syringe outlet in an open position.

2. The syringe of claim 1 wherein the syringe inlet is operable to form a first fluid coupling with a biological fluid treatment chamber outlet.

3. The syringe of claim 2 wherein the syringe outlet is operable to form a second fluid coupling with a medical accessory.

4. The syringe of claim 1 wherein the releasable lock being operable in response to a release signal to operate the syringe outlet valve between an open state and a closed state.

5. The syringe of claim 4 wherein the releasable lock is opened upon positive confirmation of the correlation between the patient identifier and the unique identifier, and provided that the at least one time period is within a predefined range.

6. The syringe of claim 5 wherein the releasable lock is operable to place the outlet valve in an irreversible closed state following the positive confirmation of the correlation between the patient identifier and the unique identifier, and provided that the at least one time period is within a predefined range.

7. The syringe of claim 3 wherein the syringe outlet includes a coupler engageable with a complementary coupler included with the medical accessory.

8. The syringe of claim 1 including a channel portion having electronic circuitry for transmitting, receiving and storing data related to the syringe and/or its contents or the patient; the circuitry comprising a transmitter, a receiver, an antenna, processor, computer readable medium, a timing circuit for maintaining temporal data related to the treatment process, a power source and input/output devices.

9. The syringe of claim 8 wherein the electronic circuitry includes an RFID tag.

10. The syringe of claim 9 wherein the RFID tag is active, semi-active or passive.

11. The syringe of claim 8 further including a recessed portion for engaging a complementary portion of a reader for acquiring the data related to the syringe and/or its contents or the patient, and the temporal data.

12. The syringe of claim 11 wherein the reader provides energizing power to the solenoid assembly, the recessed portion having electrical contacts coupled to the solenoid assembly, the electrical contacts interfacing complementary contacts associated with the reader.

13. The syringe of claim 3 wherein the releasable lock means controls the coupling of the syringe outlet coupler and the complementary coupler included with the medical accessory.

14. The syringe of claim 13 wherein the releasable lock means comprises the solenoid assembly and a locking pawl.

15. The syringe of claim 14 further including a collar intermediate the channel portion and the syringe outlet, the collar engaging the locking pawl via an opening therethrough, in the open state and a closed state.

16. The syringe of claim 1 wherein the syringe outlet valve comprises a filter in the passage for expelling one or more gas constituents in the treated sample.

17. A method for processing of a first patient material sample for administering to a second patient, comprising the steps of:
equipping the first patient with a first machine-readable patient identifier;
equipping the second patient with a second machine-readable patient identifier;
providing a first device for receiving the first patient material sample, the first device having a machine-readable first device identifier;
transferring the first patient material sample to at least one treatment device;
enabling at least one treatment device to carry out a treatment of the first patient sample material;
providing a second device for receiving the treated first patient material sample, the second device having a machine-readable machine-readable second device identifier;
providing at least one correlation unit,
recording temporal data associated with said steps of said processing, and corresponding to first patient material sample treatment events;
enabling the correlation unit to perform a correlation function between the first patient machine-readable identifier, the machine-readable second patient identifier, the machine-readable first device identifier; and the machine-readable second device identifier;
enabling the correlation unit to analyze said temporal data to determine whether said steps occur within predetermined time periods;
whereby the second device delivers the treated first patient material sample following a correlation between the first patient machine-readable identifier, the machine-readable second patient identifier, the machine-readable first device identifier; and the machine-readable second device identifier; and provided that said steps occur within predetermined time periods.

18. The method of claim 17 including further steps of providing the second device comprising:
a second device inlet operable to form a fluid coupling with a treatment device outlet;
a second device chamber for receiving the treated first patient material sample;
a second device outlet;
a passage in communication with the chamber and the second device outlet;
a releasable lock means having a biased blocking member within the passage and a solenoid assembly with a biased solenoid core engaging the blocking member to maintain the second device outlet in a closed position;
a release signal generating means for issuing a release signal to the releasable lock means upon positive confirmation of the correlation between the patient identifier and the unique identifier, and provided that the at least one time period is within a predefined range;
whereby the solenoid assembly is actuated in response to the release signal to disengage the biased solenoid core from the biased blocking member, thereby placing the second device outlet in an open position for administering into the second patient.

19. The method of claim 18 further including the step of providing electronic circuitry for transmitting, receiving and storing data related to at lease of the syringes and/or the contents or at least one of the patients; the circuitry comprising a transmitter, a receiver, an antenna, processor, computer readable medium, a timing circuit for maintaining temporal data related to the treatment process, a power source and input/output devices.

20. The method of claim 19 wherein the electronic circuitry includes an RFID tag.

21. The method of claim 20 wherein the RFID tag is active, semi-active or passive.

22. The method of claim 18 wherein at least of the devices is a syringe.

23. The method of claim 18 wherein said first patient and the second patient are the same entity.
